(19) United States
(12) Patent Application Publication
(10) Pub. No.: US 2013/0121920 A1
(43) Pub. Date: May 16, 2013

(54) METHODS FOR EVALUATING DERMAL FILLER COMPOSITIONS

(71) Applicant: ALLERGAN, INC., Irvine, CA (US)

(72) Inventors: Sumit Paliwal, Goleta, CA (US); Dennis Van Epps, Goleta, CA (US)

(73) Assignee: ALLERGAN, INC., Irvine, CA (US)

(21) Appl. No.: 13/672,490

(22) Filed: Nov. 8, 2012

Related U.S. Application Data
(60) Provisional application No. 61/558,325, filed on Nov. 10, 2011.

(51) Int. Cl. A61K 49/00

(52) U.S. Cl. CPC A61K 49/0008
USPC 424/9.2

(57) ABSTRACT

The present invention provides methods for evaluating dermal fillers for discoloration potential.

This application claims priority to U.S. Provisional Patent Application No. 61/558,325, filed Nov. 10, 2011, the entire disclosure of which is incorporated herein by this reference.

The present disclosure generally relates to methods for evaluating and selecting aesthetic dermal fillers, and more specifically relates to methods for evaluating discoloration in skin for example, as a result of Tyndall effect, of such fillers.

### BACKGROUND

Skin aging is a progressive phenomenon, occurs over time and can be affected by lifestyle factors, such as alcohol consumption, tobacco and sun exposure. Aging of the facial skin can be characterized by atrophy, slackening, and fattening. Atrophy corresponds to a massive reduction of the thickness of skin tissue. Slackening of the subcutaneous tissues leads to an excess of skin and ptosis and leads to the appearance of drooping cheeks and eye lids. Fattening refers to an increase in excess weight by swelling of the bottom of the face and neck.

Hyaluronic acid (HA), also known as hyaluronan, is a glycosaminoglycan that is distributed widely throughout the human body in connective, epithelial, and neural tissues. Hyaluronic acid is abundant in the different layers of the skin, where it has multiple functions such as, e.g., to ensure good hydration, to assist in the organization of the extracellular matrix, to act as a filler material; and to participate in tissue repair mechanisms. However, with age, the quantity of hyaluronic acid, collagen, elastin, and other matrix polymers present in the skin decreases. For example, repeated exposed to ultra violet light, e.g., from the sun, causes dermal cells to both decrease their production of hyaluronan as well as increase the rate of its degradation. This loss of materials results in various skin conditions such as, e.g., wrinkling, hollowness, loss of moisture and other undesirable conditions that contribute to the appearance of aging.

Injectable dermal fillers have been successfully used in treating the aging skin. The fillers can replace lost endogenous matrix polymers, or enhance/facilitate the function of existing matrix polymers, in order to treat these skin conditions. Hyaluronic acid-based dermal fillers have become increasingly popular, as hyaluronic acid is a substance naturally found throughout the human body. These fillers are generally well tolerated, nonpermanent, and a fairly low risk treatment for a wide variety of skin conditions.

Tyndall effect is an adverse event occurring in some patients administered with hyaluronic acid (HA)-based dermal fillers. Tyndall effect is characterized by the appearance of a blue discoloration at the skin site where a dermal filler had been injected, which represents visible hyaluronic acid seen through the translucent epidermis. Clinical reports suggest that filler administration technique and skin properties can influence the manifestation of this adverse event. Fillers with high stiffness and elasticity are successfully used to correct areas on the face like nasolabial folds, cheeks, and chin without any fear of facial discoloration, as the materials are injected in the mid and deep dermis regions. However, when these filler materials are used to correct superficial, fine line wrinkles, for example, tear trough, glabellar lines periorbital lines, smile lines, or forehead, or mistakenly applied too superficially in the upper regions of the dermis, a bluish discoloration of the skin is often observed. This phenomenon, which is thought to be the result of Tyndall effect, leaves a semi-permanent discoloration of the application sites, and sometimes disappears only after the administration of hyaluronidase to degrade the filler material. Consequently, Tyndall effect is more common in patients treated for superficial fine line wrinkles. Prolonged manifestation of Tyndall effect, typically for several months as long as the gel lasts in the skin, is a cause of major concern among patients.

Commercial dermal filler gels have been specifically formulated to treat “fine line” wrinkles found around the tear trough, forehead, periorbital, glabellar lines, etc. Many of these commercially available fine line gels show discoloration of skin, believed to be a result of Tyndall effect, particularly when injected too superficially.

Prolonged manifestation of Tyndall effect until the gel lasts in the skin (typically for several months) is a cause of major concern among patients. Despite these concerns, it is unfortunate that there are no current methods available for a priori evaluation of HA fillers to predict whether a specific filler will manifest Tyndall effect. Furthermore, no methods exist to quantify Tyndall effect and determine the performance of HA fillers. Shortage of such methods has significantly impeded the discovery process of novel fillers that will not show Tyndall effect.

It would be beneficial to have available an assay or method for facilitating development of long lasting, translucent fillers which can be injected superficially to treat fine lines and wrinkles, even in regions of relatively thin skin, without any resulting blue discoloration from Tyndall effect.

### SUMMARY

The present disclosure provides methods for evaluating dermal filler compositions, for example, for predicting manifestation of and/or quantifying, discoloration due to Tyndall effect. The present disclosure further provided methods for preparing injectable compositions having reduced Tyndall effect.

Generally, the methods generally comprise introducing a composition to be evaluated into a skin region, and measuring discoloration of the skin region. In one aspect, the discoloration measured is blue discoloration, for example, as a result of Tyndall effect.

The skin region may be a skin region of an animal, for example, a living animal, human or nonhuman. The skin region may be excised skin, freshly excised or frozen.

In one embodiment, the method includes reducing blood flow to the skin region prior to the step of measuring. For example, a vasoconstrictor may be introduced to the skin region. Alternatively or additionally, the animal is a non-human animal such as a mammal or bird. The non-human animal may be euthanized prior to the measuring in order to reduce blood flow to the skin region having the composition introduced therein. In one aspect, the blood flow is reduced within a certain defined time period following the introduction of the composition. For example, in some embodiments, the animal is euthanized no sooner than between about 12 hours and about 60 hours after the step of introducing the composition.

The skin region having the composition introduced therein, hereinafter sometimes referred to as the “treated skin” or “treated skin region”, may be compared to a untreated skin region (“untreated skin” or “untreated skin region”) for example, of the same animal.

Measurements of one or more color components of the treated skin region may be compared with such color components of untreated skin, and such comparison may be used to quantify a degree or amount of discoloration, if any, resulting from the presence of the composition in the treated skin region.

In one aspect of the invention, measuring discoloration includes measuring a blue color component of the skin, in that blue is the color of which Tyndall effect manifests in skin. Measurements may be made at space apart locations in the skin region, and the measurements averaged.

Measuring may include visually assessing, for example, visually observing and quantifying the discoloration. Alternatively or additionally, the measuring may include electromechanical measurement, assessment or observation, for example, using a suitable electromechanical instrument.

Measuring may include quantifying a blue color component of the skin region and assigning the discoloration a grade based on a numerical scale, for example, a scale of 1 to 5. Measuring may include quantifying a color component, for example, a blue color component of the skin region and comparing the blue color component of treated skin region with a blue color component of untreated skin. Quantifying a color component may include quantifying the blue color component on a l-a-b color scale, alternatively or additionally, quantifying a color component may include quantifying a percentage (%) of blue light remitted from the skin region using a suitable electromechanical instrument, for example, an spectrophotometer.

Aspects of this disclosure further include methods for evaluating a hyaluronic acid-based dermal filler for its potential for causing discoloration in skin when introduced into skin. Other aspects of this disclosure include methods for determining potential for discoloration of skin caused by a dermal filler composition being introduced therein. In some embodiments, these methods comprise introducing, for example, using linear threading technique, into a skin region of an animal, a composition to be evaluated, reducing blood flow to the skin region after the step of introducing the composition, measuring discoloration in the skin region using an electromechanical device, and comparing the measured discoloration with an area of untreated skin of the animal.

Generally, the present disclosure provides useful methods for identifying HA-based dermal fillers which will be cosmetically acceptable and will enhance the appearance of the skin without causing Tyndall effect when implanted superficially and/or into thin skin, such as the periocular region, the periorbital region or tear trough.

The present disclosure further provides useful methods for preparing HA-based dermal fillers which will be cosmetically acceptable and will enhance the appearance of the skin without causing Tyndall effect when implanted superficially and/or into thin skin, such as the periorbital region or tear trough.

### BRIEF DESCRIPTION OF THE DRAWINGS

These and other aspects of this disclosure may be more readily understood and appreciated with reference to the following description and accompanying drawings of which:

FIG. 1 shows images of skin of test animals after superficial injection of different optically transparent, HA-based dermal filler compositions;

FIG. 2A is a schematic showing anatomical locations of injections of dermal filler compositions in rats in accordance with certain aspects of this disclosure.

FIG. 2B shows steps of a linear injection technique useful in methods for evaluating dermal filler compositions;

FIG. 3 is a diagram of locations of spaced apart measurement in a treated skin region of a test mammal.

FIG. 4 shows a bar graph representing visual discoloration scores of skin regions injected with different dermal filler compositions;

FIG. 5 shows a bar graph representing blue color component (as defined by l-a-b color system) of skin regions injected with different HA-based dermal filler compositions; and

FIG. 6 shows a bar graph representing percentage (%) of blue light reflected from skin injected with different HA-based dermal filler compositions.

### DETAILED DESCRIPTION

Generally, methods for evaluating dermal filler compositions for the potential to cause discoloration, for example, Tyndall effect, in skin, are provided. The compositions which may be tested with these methods include any compositions which could potentially cause discoloration when introduced into skin, for example, human skin. Such discoloration may not be due to a physiological reaction of the body to the composition, but is sometimes a result of selective reflectance or absorbance of visible light of the composition through the skin, for example what is commonly known as “Tyndall effect”. Tyndall effect commonly manifests as a blue discoloration in thin or fair skinned individuals when certain compositions are injected too superficially. Unfortunately, Tyndall effect persists in the skin until the composition is removed from the skin, for example, by biodegradation or otherwise. Compositions exhibiting Tyndall effect include, but are not limited to, optically transparent, substantially optically transparent, biocompatible polymers, for example, polysaccharides such as crosslinked hyaluronic acid (HA)-based compositions.

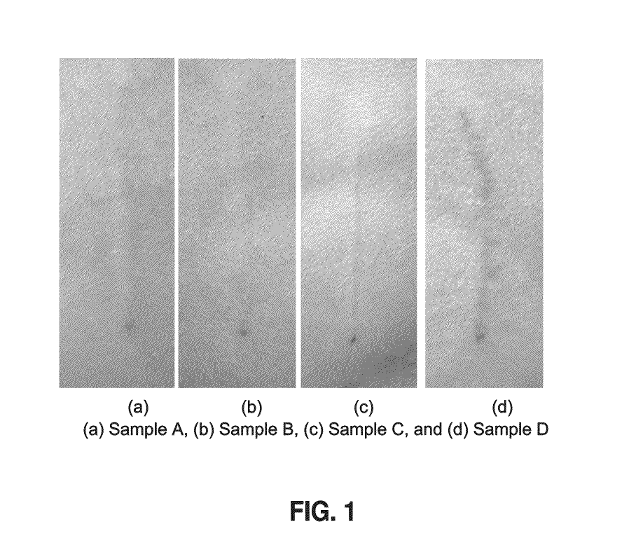

FIG. 1 shows discoloration of skin regions of four animals, in this case, hairless rats, as a result of different dermal filler compositions introduced superficially therein. Skin regions in photos c and d show marked discoloration, specifically blue discoloration, as a result of Tyndall effect. Tyndall effect, as mentioned elsewhere herein, is a significant adverse event experienced by some dermal filler patients. Development of dermal fillers that will not exhibit Tyndall effect has been problematic in the art. It is not readily apparent from the physical or chemical properties of these compositions which compositions will exhibit Tyndall effect, and which ones will not.

The skin is composed of three primary layers: the epidermis, which provides waterproofing and serves as a barrier to infection; the dermis, which serves as a location for the appendages of skin; and the hypodermis (subcutaneous adipose layer). The epidermis contains no blood vessels, and is nourished by diffusion from the dermis. The main type of cells which make up the epidermis are keratinocytes, melanocytes, Langerhans cells and Merkels cells. The dermis is the layer of skin beneath the epidermis that consists of connective tissue and cushions the body from stress and strain. The dermis is tightly connected to the epidermis by a basement membrane. It also harbors many mechanoreceptor/nerve endings that provide the sense of touch and heat. It contains the hair follicles, sweat glands, sebaceous glands, apocrine glands, lymphatic vessels and blood vessels. The blood vessels in the dermis provide nourishment and waste removal from its own cells as well as from the Stratum basale of the epidermis. The dermis is structurally divided into two areas: a superficial area adjacent to the epidermis, called the papillary region, and a deep thicker area known as the reticular region.

Fine lines or superficial wrinkles are generally understood to be those wrinkles or creases in skin that are typically found in regions of the face (forehead, lateral canthus, vermillion border/perioral lines) where the skin is thinnest, that is, the skin has a dermis thickness of less than 1 mm. On the forehead the average dermal thickness is about 0.95 mm for normal skin and about 0.81 mm for wrinkled skin. Dermis around the lateral canthus is even thinner (e.g. about 0.61 mm for normal skin and about 0.41 mm for wrinkled skin). The average outer diameter of a 30 or 32 gauge needle (needles that are typically used for fine line gel application) is about 0.30 and about 0.24 mm.

Tyndall effect often manifests in patients treated for superficial fine line wrinkles. Thus, aspects of this disclosure provide assays and methods for facilitating development of long lasting, translucent fillers which can be injected superficially to treat fine lines and wrinkles, even in regions of relatively thin skin, without any resulting blue discoloration from Tyndall effect. Methods for evaluating compositions and identifying those which will not exhibit significant discoloration, for example, from Tyndall effect, when the compositions are used to treat fine lines, are provided.

Compositions with reduced risk of discoloration when introduced into skin may be identified in accordance with the present disclosure, wherein such compositions are dermal fillers useful for treating various skin conditions. Compositions may be identified which are useful for fine line treatment of skin, for example, for reducing the appearance of superficial, relative shallow wrinkles in skin, for example, thin skin, of a human being. Compositions may be identified which are useful for treating skin dehydration, wherein such composition, when introduced into the skin, rehydrates the skin, thereby treating skin dehydration, without undesirably discoloring the appearance of the skin. Compositions may be identified which are useful for treating lack of, or reduced, skin elasticity, wherein such composition, when introduced into the skin, increases the elasticity of the skin, without undesirably discoloring the appearance of the skin. Compositions may be identified which are useful for treating skin roughness wherein such composition, when introduced into the skin, decreases skin roughness, without undesirably discoloring the appearance of the skin. Similarly, compositions may be identified which are useful for treating lack of or reduced skin tautness, wherein such composition, when introduced into the skin, makes the skin tauter, without undesirably discoloring the appearance of the skin.

Accordingly, the present disclosure provides methods for evaluating compositions for their potential to cause discoloration before the compositions are used in a therapeutic or cosmetic setting in human beings.

In one embodiment, a method for evaluating a dermal filler composition comprises introducing into a skin region, a composition to be evaluated, and measuring discoloration of the skin region.

The skin region may be a skin region of an animal, for example, a living animal, human or nonhuman. In some embodiments, the animal is a living non-human mammal, such as a hairless rat. Other useful test animals will be known to those of skill in the art. In some embodiments, the skin region is excised skin, freshly excised skin or previously frozen skin.

As used herein, the terms “skin region” and “dermal region” refers to the region of skin comprising the epidermal-dermal junction and the dermis including the superficial dermis (papillary region) and the deep dermis (reticular region).

The method may further comprise the step of reducing blood flow to the skin region. Although discoloration from Tyndall effect may manifest sometimes immediately after introduction of the composition into the skin region, the blue discoloration becomes more pronounced over the next couple of days.

In some embodiments, the discoloration is observed and/or measured after a selected time period has passed to enable more pronounced manifestation of the discoloration. The time period may be, for example, after at least twelve hours, at least twenty four hours, at least thirty six hours, at least forty eight hours, or longer after introduction of the composition into the skin region.

In some embodiments, blood flow is reduced to the skin region, for example, before or after the introduction of the composition to the skin region. In some embodiments the step of reducing blood flow is performed after the selected time period.

Reducing blood flow may be accomplished by any suitable means, for example, by applying or introducing a suitable vasoconstrictor, or vasoconstriction agent to the skin region. Reducing blood flow may further or alternatively be accomplished by reducing the temperature of the skin region, for example, by application of a cold compress or ice to the region.

In some embodiments, the reduced blood flow is accomplished by euthanizing the animal. Euthanizing may lead to significant improvement in the contrast of Tyndall effect, for example, on rat skin. Improvement in contrast is possibly due to lack of hemoglobin in skin. Hemoglobin provides a strong red color tone to hairless rat skin which is notably higher than the typical red color tone in human skin. Therefore, for model purposes, euthanizing the animal for observing Tyndall effect provides a good clinical relevance.

The step of measuring the discoloration may comprise visually assessing the discoloration, for example, visually observing the discoloration by comparing the treated skin region with an untreated skin region. The discoloration may be assigned a grade based on a numerical scale, for example, a scale of 1 to 5 in increments of 0.5. A score of 1 may be assigned to treated skin regions with normal skin tone and no blue discoloration, such as shown in FIG. 1, photo (b). A maximum score of 5 may be assigned to thick and pronounced blue discoloration, such as shown in FIG. 1, photo (d).

In another aspect, the measuring comprises assessing the discoloration using an electromechanical device. For example, the measuring comprises assessing the discoloration using instruments used to measure color using reflectance spectroscopy, such as a spectrophotometer. A color component of the treated skin region may be quantified in any suitable manner. In some embodiments, the method comprises quantifying a percentage (%) of blue light remitted from the skin region.

In an aspect of this disclosure, the color component measured, for example, the blue color component, may be quantified based on the L-a-b color scale. Unlike other color models, the L-a-b color scale is designed to approximate human vision. Its “L” component closely matches human perception of lightness. Instruments useful for accurately measuring color are available, including instruments referred to as spectrophotometer or chromameter.

To increase accuracy of the assay, the color component may be measured or quantified as described, at a plurality of separate spaced apart locations in the skin region and the measurements averaged. Further, the measurements may be compared to measurements of color of untreated skin.

### Example 1

### Method of Evaluating Discoloration

The following example describes a method for reproducibly assessing skin discoloration after dermal filler administration, such as discoloration due to Tyndall effect. The method is demonstrated in rats, as an example; however, it should be appreciated that the method is not limited in its applicability to other animals, for example, mammals or avian species.

Anesthetize a 2 month old hairless rat using induction chamber maintained under 4% isofluorane and oxygen. Transfer the animal to an operating table and maintain a constant flow of anesthesia at 2-2.5% isofluorane and oxygen.

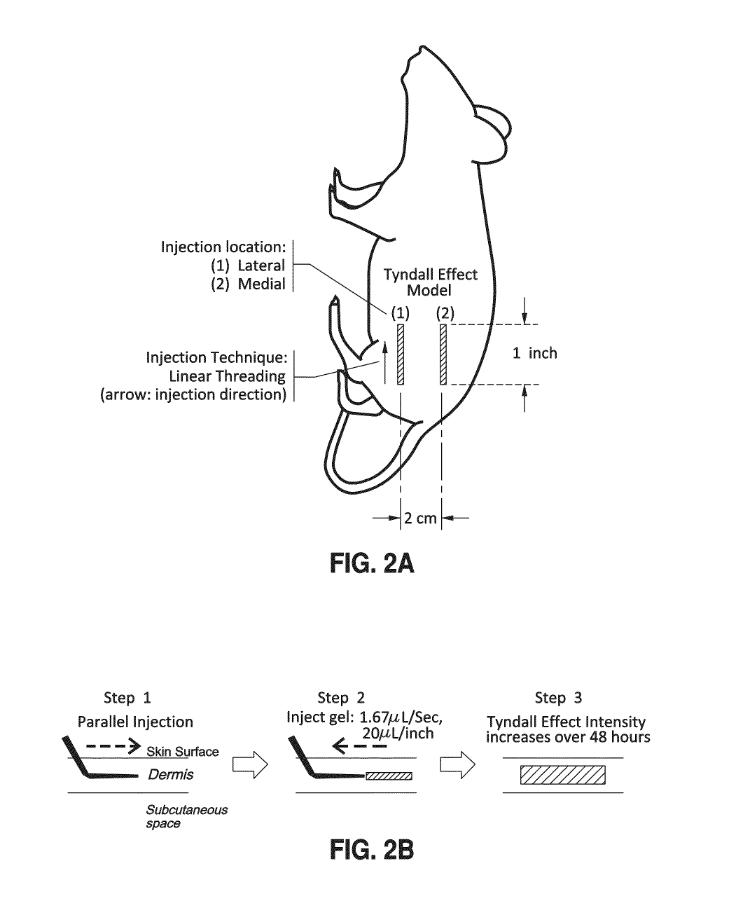

Place the animal in lateral position exposing flank region (see FIG. 2A). Lightly shave hair on skin using a clipper. Avoid damage to skin from the blades of the clipper.

Prepare filler injection apparatus. In this example, the injection apparatus is a syringe pump connected to a 1″ long 27G needle using a 13″ long sterile silicone cannula. Syringe pump is calibrated to inject filler at a rate of 100 μL/min from the syringe with internal diameter of 4.6 mm.

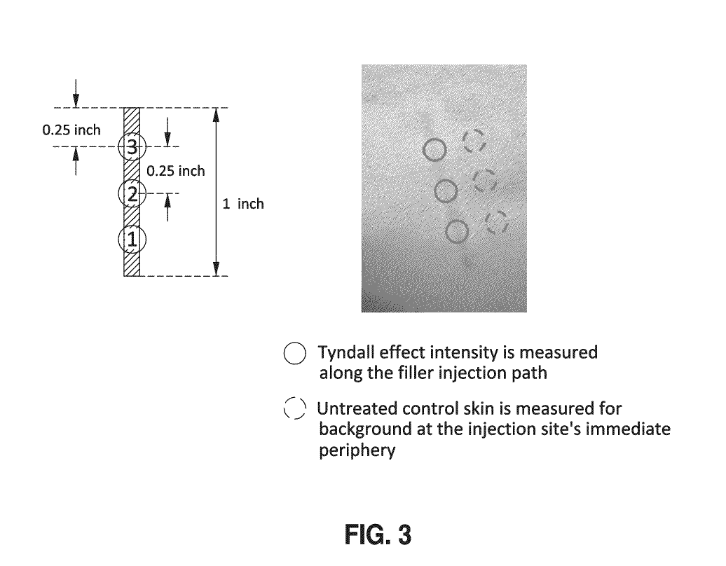

Filler injections are performed parallel to the anteroposterior axis using a linear threading technique (see FIG. 2B). Samples are placed intradermally in the lateral-to-midline and caudal to ribs region (refer to FIG. 2A). Needle is inserted in skin from the posterior end of the animal. Once inside the dermis, the needle is fully inserted parallel to skin surface towards the anterior direction. Start the syringe pump and wait for 13 seconds (syringe pump displays 22 μL as injected volume). This is the “ramp-up time” taken for the viscous gel to start evenly flowing from the needle. At this time, start retracting the needle smoothly and evenly laying the gel along the injection path. Time the retraction rate such that about 20 μL of filler is injected along the 1″ long injection path. Stop the syringe pump. The Tyndall effect starts to develop over time at the filler injection site (Step 3, FIG. 3).

The injections can be (optionally) performed at two locations per lateral flank side of the animal—(1) ventral, and (2) dorsal (refer to FIG. 1). If performing 2 injections, separate the injections at the two locations by at least 2 cm.

Discoloration effect is measured 48 hours post injection after euthanizing the animals. Euthanizing the animals eliminates strong red color tone of skin and makes Tyndall effect more pronounced.

Discoloration intensity for each injected filler site can be quantified visually and/or using spectroscopy:

### Method 1. Visual Measurement

A Tyndall Effect Visual Score is defined by visually assessing the blue discoloration at the injection site compared to the adjacent untreated skin. The scale has a range of 1 to 5 with increments of 0.5. A score of 1 is given to injection sites with normal skin tone and no blue discoloration. A maximum score of 5 is given to thick and pronounced blue discoloration (typically associated with certain commercial HA based fillers). Three independent observers are trained on the scale before being blinded to score test samples.

### Method 2. Spectroscopy Measurement

Reflectance spectroscopy is used to quantitatively assess the blue color of skin. Using this technique two distinct parameters can be defined that independently measure the intensity of blue color in skin, or Tyndall effect. These parameters are described below:

### Method 2.1. Blue Component of Skin Color—“b”

A chromameter is used to quantify the blue color component of light remitted from skin sites injected with the various fillers. This is achieved by using the “b” component of L-a-b color scale. The L-a-b color scale uses 3 component (L, a and b) notation that can be used to define any color and is designed to approximate human vision. L defines lightness, and a and b define color-opponent dimensions. Specifically, component b defines color varying from yellow (positive axis) to blue (negative axis). A highly negative “b” component for skin color will mean skin has a strong blue discoloration, as seen in Tyndall effect. L-a-b color aspires to perceptual uniformity, and its L component closely matches human perception of lightness. It can thus be used to make accurate color balance corrections by modifying output curves in the a and b components, or to adjust the lightness contrast using the L component.

### Method 2.2. “% Blue Light” Remitted from Skin

A portable spectrophotometer is be used to quantify the % blue light reflected from skin in the total visible light range.

Spectrophotometer measures the visible light reflected from skin surface, specifically between 400-700 nm wavelengths. The % blue light reflected from skin can be quantified by integrating the area under the visible light spectrum between 400-490 nm and normalizing it by the total area under the spectrum (400-700 nm). An increase in the % blue light as determined from the reflected light spectrum will mean skin has a strong blue discoloration, as seen in Tyndall effect.

Based on spectroscopic quantification of discoloration, e.g. due to Tyndall effect, each injection site is measured at three equidistant locations along the injection path (see FIG. 3). For a 1″ injection path, each of the measured locations are 0.25″ apart such that the first measured location is positioned 0.25″ from the injection entry location. Finally, the two Tyndall effect intensity parameters ((a) blue component of skin color—“b”, and (b) “% Blue Light” remitted from skin) are calculated for each location. The measurements are further averaged for the three locations along the injection path to calculate a single measurement for the injected skin site. Similar measurements are performed on untreated skin adjacent to the injection site to measure background (untreated skin) discoloration.

By using the method described herein, the intensity of discoloration, for example, from Tyndall effect, and the potential for manifestation of Tyndall effect, in human skin, for example can be evaluated for a particular filler composition.

### Example 2

### Visual Assessment and Quantification of Skin Discoloration

FIG. 1 shows different levels of visually apparent discoloration in rats after superficial injection of four different HA-based compositions: Sample A, Sample B, Sample C and Sample D. The samples differ from one another based on, for example, concentration of hyaluronic acid, the inclusion or absence of additives, degree of crosslinking, etc. In this example, each of the samples is substantially entirely clear or optically transparent prior to being placed in the skin. However, as can be appreciated from viewing FIG. 1, the samples manifest blue discoloration, presumably as a result of Tyndall effect, to varying degrees.

The blue discoloration is measured visually and spectroscopically as described elsewhere herein.

### Example 3

### Visual Assessment and Quantification of Discoloration in Cadaver Skin

Samples of different HA-based fillers are implanted superficially in freshly excised cadaver skin. Visual and spectroscopic analysis is performed such as described in Example 1. Some of the samples manifest blue discoloration, presumably as a result of Tyndall effect. Other of the samples do not manifest blue discoloration.

The blue discoloration is measured visually and spectroscopically as described elsewhere herein.

### Example 4

### Visual Assessment and Quantification of Discoloration in Avian Skin

Samples of different HA-based fillers are implanted superficially in previously frozen chicken skin. Visual and spectroscopic analysis is performed such as described in Example 1. Some of the samples manifest blue discoloration, presumably as a result of Tyndall effect. Other of the samples do not manifest blue discoloration.

The blue discoloration is measured visually and spectroscopically as described elsewhere herein.

### Example 5

### Quantitative Assessment of Skin Discoloration by Dermal Fillers

Two months old hairless rats were intradermally injected using linear threading technique with four different HA-based dermal filler compositions: Sample A, Sample B, Sample C and Sample D (as shown in FIG. 1 and discussed in Example 2). In order to test Tyndall effect, the gels were placed superficially to match the clinical procedure for treating fine lines. The appearance of the gel in skin was followed for 2 days. Although Tyndall effect manifested immediately after gel injection, the blue discoloration became more pronounced over next couple of days. Forty eight hours after gel implantation, the animals were euthanized leading to a significant improvement in the contrast of Tyndall effect on rat skin. In order to compare the performance of these fillers, a quantitative analysis of Tyndall effect was performed as follows.

Tyndall Effect Visual Score:

The scale had a range of 1 to 5 with increments of 0.5. A score of 1 was given to injection sites with normal skin tone and no blue discoloration. A maximum score of 5 was given to thick and pronounced blue discoloration (typically associated with Restylane or Juvéderm Ultra Plus). Three independent observers were trained on the scale before being blinded to score test samples.

Blue Component of Skin Color—“b”:

A chromameter (CM2600D, Konica Minolta, NJ) was used to quantify the blue color component of light remitted from skin sites injected with the various fillers. This was achieved by using the “b” component of L-a-b color scale.

“% Blue Light” Reflected from Skin:

A portable spectrophotometer (CM2600D, Konica Minolta, NJ) was used to quantify the % blue light remitted from skin in the total visible light range. This was achieved by integrating the area under the visible light spectrum between 400-490 nm and normalizing it by the total area under the spectrum (400-700 nm).

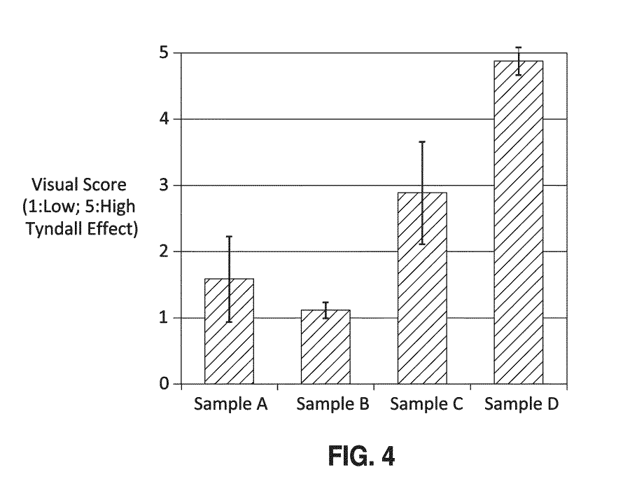

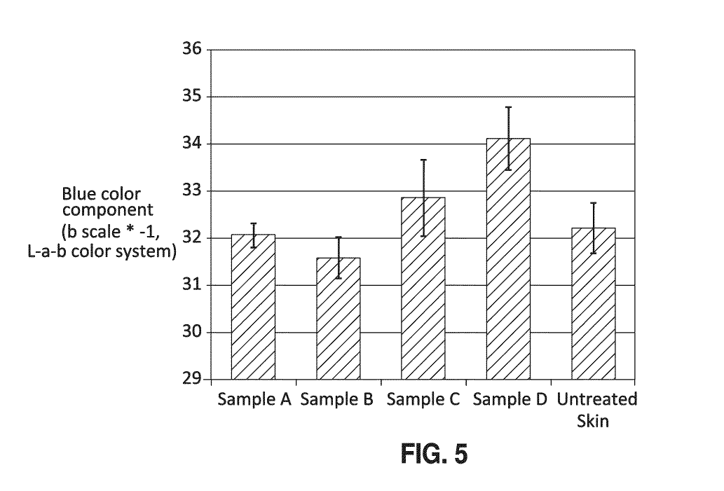

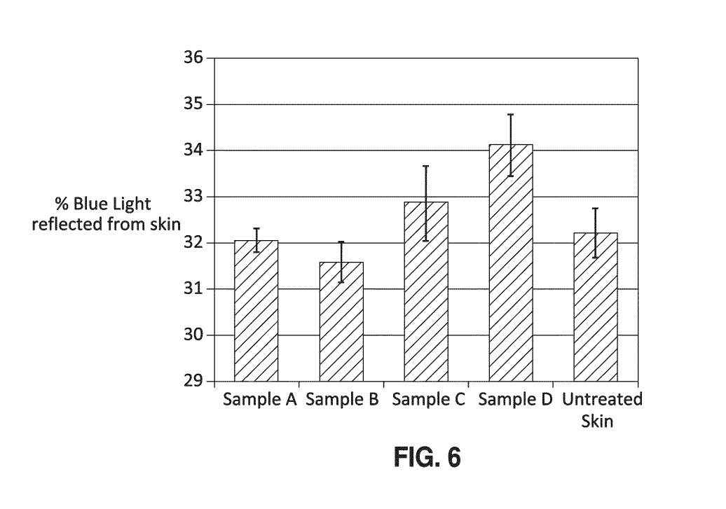

FIG. 4 reports Tyndall effect visual scores (method a) for various HA gels. FIG. 5 reports spectrophotometric analysis by measuring blue color component (method b) for each filler's injection site. FIG. 6 reports spectroscopic measure of % blue light reflected from skin (method c). All of these parameters successfully differentiated different filler types in terms of their ability to produce skin discoloration due to Tyndall effect. For example in FIG. 5 and FIG. 6, Samples A and B showed lower blue color component and % blue light values than untreated skin control signifying that the Sample will have substantially or entirely no perceptible discoloration due to Tyndall effect. In contrast, Samples C and D showed significantly higher blue color component and % blue light values than untreated skin control signifying the presence of discoloration due to Tyndall effect.

In closing, it is to be understood that although aspects of the present specification have been described with reference to the various embodiments, one skilled in the art will readily appreciate that the specific examples disclosed are only illustrative of the principles of the subject matter disclosed herein. Therefore, it should be understood that the disclosed subject matter is in no way limited to a particular methodology, protocol, and/or reagent, etc., described herein. As such, those skilled in the art could make numerous and various modifications or changes to or alternative configurations of the disclosed subject matter can be made in accordance with the teachings herein without departing from the spirit of the present specification. Changes in detail may be made without departing from the spirit of the invention as defined in the appended claims. Lastly, the terminology used herein is for the purpose of describing particular embodiments only, and is not intended to limit the scope of the present invention, which is defined solely by the claims. In addition, it is intended that all matter contained in the above description or shown in the accompanying drawings shall be interpreted as illustrative only and not limiting. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

What is claimed is:

1. A method for evaluating a dermal filler composition, the method comprising:
introducing into a skin region a dermal filler to be evaluated; and
measuring discoloration in the skin region.

2. The method of claim 1 wherein the skin region is a skin region of a living animal.

3. The method of claim 1 wherein the skin region is a skin region of a living animal and the method further comprising reducing blood flow to the skin region prior to the step of measuring.

4. The method of claim 3 wherein the step of reducing blood flow comprises introducing a vasoconstrictor to the skin region.

5. The method of claim 3 wherein the step of reducing blood flow comprises euthanizing the animal.

6. The method of claim 5 wherein the euthanizing is performed at least 12 hours after the step of introducing.

7. The method of claim 5 wherein the euthanizing is performed at least 36 hours after the introducing.

8. The method of claim 5 wherein the euthanizing is performed at least 48 hours after the introducing.

9. The method of claim 1 wherein the observing is performed substantially immediately after the step of introducing into a skin region.

10. The method of claim 1 wherein the discoloration measured is a blue discoloration.

11. The method of claim 1 wherein the discoloration measured is discoloration resulting from Tyndall effect.

12. The method of claim 1 wherein the measuring comprises visually assessing the discoloration.

13. The method of claim 1 wherein the measuring comprises visually assessing the discoloration and assigning the discoloration a grade based on a numerical scale.

14. The method of claim 1 wherein the measuring comprises assessing the discoloration using an electromechanical device.

15. The method of claim 14 wherein the measuring comprises assessing the discoloration using reflectance spectroscopy.

16. The method of claim 1 wherein the measuring comprises quantifying a blue color component of the skin region.

17. The method of claim 1 wherein the measuring comprises quantifying a percentage (%) of blue light remitted from the skin region.

18. The method of claim 1 wherein the measuring comprises quantifying a blue color component of the skin region using an electromechanical device.

19. The method of claim 1 wherein the measuring comprises quantifying a blue color component of the skin region using a spectrophotometer.

20. The method of claim 1 wherein the measuring comprises measuring discoloration at a plurality of separate spaced apart locations in the skin region.

21. The method of claim 1 wherein the measuring comprises measuring discoloration at a plurality of locations in the skin region and averaging the measurements.

22. The method of claim 1 wherein the dermal filler is a hyaluronic acid-based dermal filler.

23. The method of claim 1 wherein the dermal filler is a substantially optically transparent, hyaluronic acid-based dermal filler.

24. A method for evaluating a hyaluronic acid-based dermal filler for its potential for causing discoloration in skin when introduced into skin, the method comprising:
introducing, using linear threading technique, into a skin region of an animal, a composition to be evaluated;
reducing blood flow to the skin region after the step of introducing the composition; and
measuring discoloration in the skin region using an electromechanical device;
comparing the measured discoloration with an area of untreated skin of the animal.

25. The method of claim 24 wherein the step of measuring comprises measuring a blue color component of the skin region.

26. A method for determining potential for discoloration of skin caused by a dermal filler composition being introduced therein, the method comprising:
introducing, using linear threading technique, into a skin region of an animal, a composition to be evaluated;
measuring discoloration in the skin region using an electromechanical device;
comparing the measured discoloration with an area of untreated skin of the animal.
